(19) United States
(12) Patent Application Publication
(10) Pub. No.: US 2007/0117092 A1
(43) Pub. Date: May 24, 2007

(54) Dna analysis system

(76) Inventors: Daksh Sadarangani, Auckland (NZ); Bruce Macdonald, Auckland (NZ); Paul Calverley, Auckland (NZ); Allen Rodrigo, Auckland (NZ); David Saul, Auckland (NZ); Scott Baker, Auckland (NZ); Mike Pearson, Auckland (NZ)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/552,159

(22) PCT Filed: Mar. 23, 2004
(86) PCT No.: PCT/NZ04/00057

§ 371 Date: Dec. 8, 2006

(30) Foreign Application Priority Data
Apr. 8, 2003 (NZ) 525198
Aug. 13, 2003 (NZ) 527519

(51) Int. Cl. C12Q 1/68; C12M 1/34

(52) U.S. Cl. 435/6; 435/287.2

(57) ABSTRACT

A DNA analysis system 10 includes a thermal cycler 12 operable as an extraction stage for extracting DNA from a sample to be tested and an as amplification stage for replicating identically a region of interest in DNA strands extracted from the sample. A predetermined proteinase is used in the thermal cycler 12 at least in the extraction stage. A purification stage 22 purifies the amplified material from the thermal cycler 12. An analysis stage 88 analyses the purified sample to obtain genetic information relating to the sample.

### FIELD OF THE INVENTION

This invention relates to DNA analysis. More particularly, the invention relates to a DNA analysis system and method.

### BACKGROUND TO THE INVENTION

With the identification of the structure of DNA, research and development in the field of genetics at a molecular level was established.

To analyse DNA from a sample or organism traditionally requires many different steps. It also requires at least three different items of equipment excluding the equipment used to display the result. The use of three separate automated instruments to perform different parts of the analysis process renders the equipment bulky and unable to be used in the field. Also, because the instruments are so large, it would not achieve any useful purpose to integrate them into a single unit or system. In addition, the equipment requires substantial technical expertise to operate. Therefore, most of these instruments are built for use in laboratories. A sample that requires analysis must be collected at the site and sent to the laboratory. This can, in certain circumstances, be undesirable such as, for example, at a crime scene where delays in obtaining information can lead to loss of valuable time in investigating the matter.

Still further, in the preparation of the sample for analysis purposes, a quantity of the sample is placed in a test tube which needs to be sealed and opened at intervals to add agents. Certain of these agents, apart from being toxic, need to be removed prior to analysis to inhibit contamination. Also, the need continuously to open and close the test tube containing the sample renders the sample vulnerable to being contaminated which can adversely affect the final result.

### SUMMARY OF THE INVENTION

Broadly, according to a first aspect of the invention, there is provided a DNA analysis system which includes a unit that effects both extraction of DNA and amplification by identical replication of a region of interest of extracted DNA strands, with a proteinase, as defined, being used in the unit at least to effect extraction of DNA.

The system may be used for detecting the presence of predetermined sequences such as pathogens. For this purpose, the amplification may include nucleotide sequence detection for the purpose of looking for specific sequences of DNA associated with certain pathogens, etc. Nucleotide sequence detection may therefore be performed during the amplification stage, by adding fluorescently labelled oligonucleotides that can target any specific short sequence of DNA. The unit used in this case may include an attached fluorimeter and light source.

More specifically, according to a first aspect of the invention, there is provided a DNA analysis system which includes:

a thermal cycler operable as an extraction stage for extracting DNA from a sample to be tested and as an amplification stage for replicating identically a region of interest in DNA strands extracted from the sample, a proteinase, as defined, being used in the thermal cycler at least in the extraction stage;

a purification stage for purifying the amplified material from the thermal cycler; and

an analysis stage for analysing the purified sample to obtain genetic information relating to the sample.

The use of the thermal cycler both for the extraction stage and the amplification stage may be facilitated by the use of a non-specific thermophilic enzyme as the proteinase, the thermophilic enzyme being stable and active in a temperature range of about 65-80° C. but which is denatured at a temperature exceeding about 90° C. More particularly, the proteinase used in the system is described in greater detail in International Patent Application No. PCT/NZ02/00093 to The University of Waikato. The contents of that patent application are incorporated herein by reference. The term “proteinase” as used in this specification is therefore to be understood, unless the context clearly indicates otherwise, as a proteinase having the properties as described above.

The analysis stage may comprise a separation stage and a detection stage. The system may include a sequencing stage preceding the analysis stage. The thermal cycler may also be used for the sequencing stage. Thus, one piece of equipment, being the thermal cycler, may be used for extraction, amplification and sequencing. Also, due to the fact that the proteinase is denatured during the extraction phase, the need for a centrifuge to separate out impurities from the sample is obviated.

The purification stage may incorporate a size filtration matrix comprising a gel filtration media incorporating a filtering resin, the matrix allowing larger fragments of DNA through from the amplification stage before any smaller fragments and other unwanted substances. The larger fragments may be collected for use in the sequencing stage.

The sequencing stage may tag ends of the fragments with dideoxynucleoside triphosphates (ddNTP's) labelled with different fluorochromes before grading. The grading may form the first step of the separation stage arid incorporates separating the fragments into fragments of differing lengths by a separation device.

The separation device may be an electrophoresis device. Preferably the electrophoresis device is a capillary electrophoresis device and includes a detector for detecting information relating to tagged fluorescent nucleotides at the end of each of the DNA fragments. The detector may include a laser device that irradiates the ends of the DNA fragments to cause the fluorescent ends to fluoresce.

Further, the system may include a reader for reading the fluorescent ends of the fragments. The reader may be in the form of a charge coupled device (CCD) camera or a photomultiplier tube (PMT), the output of which is fed to the monitoring means.

The thermal cycler may includes a controller which controls the various stages of preparation of the sample. In addition, the thermal cycler may include a heating mechanism for heating the sample, contained in one or more vials or test tubes, received in the thermal cycler. The heating mechanism may be controlled by the microcontroller to maintain the sample at the required temperatures at the various stages of extraction, amplification and sequencing.

The system may include a dispensing device for depositing the material to be analysed in the thermal cycler. The dispensing device may be a pipette.

Because the thermal cycler is used for various stages in the analysis procedure, it is necessary that efforts be made to minimise contamination of the sample being analysed. Accordingly, the thermal cycler may include a holder for holding replacement tips for the dispensing device. The holder may be arranged on the thermal cycler adjacent the heating mechanism within reach of the range of movements of the dispensing device.

Still further, it may be convenient to arrange the various solutions to be used in the various stages that use the thermal cycler within reach of the range of movement of the dispensing device. Thus, the holder may include reservoirs for various solutions adjacent the replacement tips. Instead, the tips may be arranged on one side of the heating mechanism and the reservoirs may be arranged on an opposed side of the heating mechanism.

In addition, the purification stage may also be mounted on the holder adjacent the heating mechanism of the thermal cycler.

The system may include a monitoring means for monitoring the analysis stage. The monitoring means may be in the form of a computer having a display on which data relating to the analysed sample are displayed.

Broadly, according to a second aspect of the invention, there is provided a method of preparing a sample for DNA analysis, the method including the step of using a single unit to effect both extraction of DNA and amplification by identical replication of a region of interest of extracted DNA strands, with a proteinase, as defined, being used in the unit at least to effect extraction of DNA.

The method may include the step of looking for specific sequences such as those associated with predetermined pathogens, etc. during amplification by including nucleotide sequence detection in the amplification stage. Thus, the method may include performing nucleotide sequence detection during amplification by adding fluorescently labelled oligonucleotides that can target a specific short sequence of DNA. The method may include using a thermal cycler that has an attached fluorimeter and light source.

More specifically, according to a second aspect of the invention, there is provided a method of preparing a sample for DNA analysis, the method including the steps of:

placing a sample of material to be analysed in a thermal cycler and adding a predetermined quantity of proteinase to the thermal cycler;

cycling the mixture through a predetermined temperature profile to effect extraction of DNA material from the sample;

in the thermal cycler, subjecting the extracted DNA material to an amplification stage replicating identically a region of interest in the extracted DNA material; and

sequencing the amplified material.

The method may include sequencing the material by a dideoxy method of sequencing which includes the steps of sequencing, separation and detection.

The method may include, as part of separating the DNA material, purifying the material and sequencing the purified DNA material. In particular, the method may include effecting the sequencing of the purified DNA material for separation and detection using the thermal cycler.

The method may include purifying the material by passing the material through a size filtration matrix comprising a gel filtration media incorporating a filtering resin, the matrix allowing larger fragments of DNA through from the amplification stage before any smaller fragments and other unwanted substances. Thereafter, the method may include collecting the larger fragments for use in the sequencing of the material.

The method may include tagging ends of the fragments with dideoxynucleoside triphosphates (ddNTP's) labelled with different fluorochromes before grading. The grading may form the first step of the separation stage and the method may incorporate separating the fragments into fragments of differing lengths.

The method may include detecting information relating to tagged fluorescent nucleotides at the end of each of the DNA fragments. Thus, the method may include irradiating the ends of the DNA fragments to cause the fluorescent ends to fluoresce and reading the fluorescent ends of the fragments.

According to a third aspect of the invention, there is provided a purification stage for a DNA analysis system, the purification stage including

a conduit; and

a gel filtration medium contained in the conduit, the gel filtration medium being a resin of microscopic, synthetic beads.

More particularly, the gel filtration medium may be of microscopic beads synthetically derived from a polysaccharide dextran.

The purification stage may include a control device for controlling the passage of the sample through the conduit. In this regard, it will be appreciated that the sample is contained in solution which is fed through the gel filtration medium. The control means may be in the form of a control valve arranged in the conduit.

According to a fourth aspect of the invention, there is provided a method of purifying a DNA sample, the method including the step of passing the sample through a conduit containing a gel filtration medium in the form of a resin of microscopic, synthetic beads to effect purification of the sample.

The method may include forming the beads from a polysaccharide.

Further, the method may include controlling the passage of the sample through the conduit.

According to a fifth aspect of the invention, there is provided a DNA analysis system which includes:

a unit operable at least as an extraction stage for extracting DNA from a sample to be tested and as an amplification stage for replicating identically a region of interest in DNA strands extracted from the sample;

a microfluidic device mounted on the unit and defining a plurality of wells interconnected by a channel, a sample undergoing various stages of preparation being moved sequentially from one well to another via the relevant interconnecting channel; and

a control arrangement for controlling movement of the sample between said wells.

The unit may also operate as a sequencing stage.

Further, the control arrangement may include an electric field generating means that moves a charged solution between the wells through the channels. The electric field generating means may comprise a plurality of electrodes, each of said predetermined wells having an electrode associated with it.

At least certain of the wells may operate as waste wells in which waste material, separated out from the sample, is deposited for disposal.

The system may include a dispensing arrangement for depositing reagents in the wells. The dispensing arrangement may comprise at least one pipette for dispensing the reagents. The pipettes may be carried on a heat control lid of the thermal cycler.

According to a sixth aspect of the invention, there is provided a method of preparing a sample for DNA analysis, the method including the steps of:

placing a sample of material to be analysed in a first well of a microfluidic device having a plurality of wells interconnected by channels;

effecting a first preparatory stage in the first well of the device;

controlling movement of the sample from one well, sequentially, to further wells in the microfluidic device and carrying out further preparatory stages at each of predetermined wells in the device.

The method may include modifying an existing thermal cycler by mounting the microfluidic device on the thermal cycler. The thermal cycler may need to be altered to perform the necessary temperature cycling reactions within the wells of the microfluidic device.

The method may include controlling the movement of the sample from well to well by means of an electric field generating means that moves a charged solution between the wells through the channels. Thus, the method may include associating an electrode with each well and controlling the movement of the sample between wells by changing the potential of the wells relative to one another.

The method may include designating one of the wells as a waste well and depositing waste material, separated out from the sample, in the waste well.

### BRIEF DESCRIPTION OF THE DRAWINGS

Embodiments of the invention are now described by way of example with reference to the accompanying diagrammatic drawings in which:

FIG. 1 shows a schematic representation of a DNA analysis system, in accordance with a first embodiment of the invention;

FIG. 2 shows a time-based schematic depiction of the operation of the system;

FIG. 3 shows a schematic representation of a DNA analysis system, in accordance with a further embodiment of the invention;

FIG. 4 shows a schematic representation of a DNA analysis system, in accordance with yet a further embodiment of the invention; and

FIG. 5 shows a schematic plan view of a microfluidic device for use with the system of FIG. 4.

### DETAILED DESCRIPTION OF THE DRAWINGS

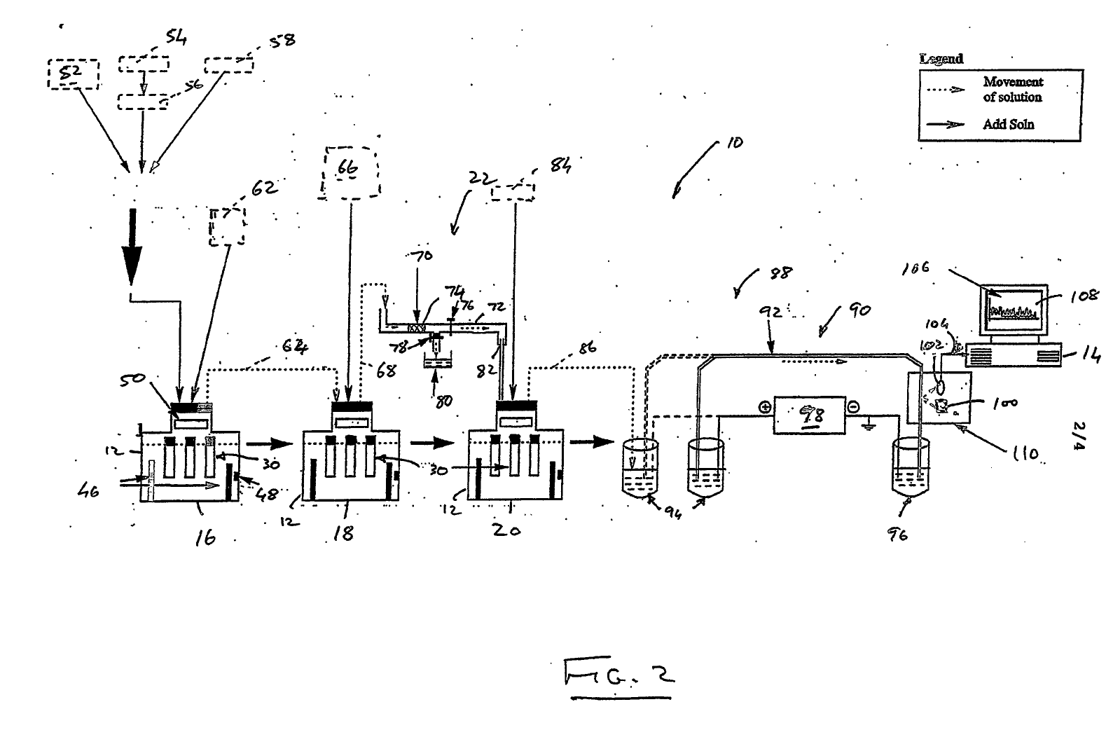

In the drawings, a DNA analysis system, in accordance with an embodiment of the invention is illustrated and is designated generally by the reference numeral 10. The system 10 includes a thermal cycler 12 and a monitoring means in the form of a computer 14. As illustrated more clearly in FIG. 2 of the drawings and as will be described in greater detail below, the thermal cycler 12 is used, initially, for an extraction stage 16 followed by an amplification stage 18 followed by a sequencing stage 20.

A purification stage 22 is interposed between the amplification stage 18 and the sequencing stage 20. It is emphasised that, what is illustrated in FIG. 2 of the drawings, is a time-based illustration of the sequence of events leading to analysis of DNA material. The thermal cycler 12 is used for all three of the extraction stage 16, the amplification stage 18, and the sequencing stage 20.

The thermal cycler 12 has a housing 24 on which a keypad 26 for controlling operation of the thermal cycler 12 is mounted. A receptacle 28 containing a plurality of reservoirs (or wells) 30, in which sample material is received, is mounted on top of the housing 24. The receptacle 28 is closed by a heat control lid 32.

A remote controlled pipette 34 is mounted on an arm 36. The pipette 34 is used to inject sample material into the reservoirs 30. The arm 36 is suspended from a beam 38. The arm 36 is displaceable horizontally along the beam 38 as indicated by arrow 40 under control of the computer 14 as illustrated by control line 42. In addition, the pipette 34 can also move vertically on the arm 36 as indicated by arrow 44, once again, under control of the computer 14.

As illustrated in FIG. 2 of the drawings, the thermal cycler 12 includes a plurality of heating elements 46 and thermocouple 48.

In use, a sample 60 of material to be analysed is inserted into one or more of the reservoirs 30 of the thermal cycler 12. The sample could be a bacterial or cultural swab 52, human or animal tissue 54 which has been homogenised as shown at 56, or human or animal blood 58. For ease of explanation, the sample will be referred to by reference numeral 60.

The sample 60 is inserted into the thermal cycler 12 together with an extraction solution 62.

The extraction solution 62 comprises proteinase as defined above. 1 μl of proteinase is added together with each unit of sample material 60. The extraction solution 62 further comprises 100 μl of buffer for each microlitre of proteinase.

The solution in the reservoirs 30 of the thermal cycler 12 is then subjected to 15 minutes of heating at about 75° C. At this temperature, the cells of the sample material 60 are lysed to facilitate extraction of DNA material. Once the DNA material has been extracted from the cells of the sample material 60, the proteinase is denatured by subjecting the solution to heat at about 95° C. for a further 15 minute period.

Approximately 1-5 μl of extracted material in solution 64 is then subjected to the amplification stage 18. The amplification stage 18 is a polymerase chain reaction (PCR) amplification stage for effecting rapid replication of a specific region of the DNA material. The solution 64 may be diluted, if necessary, so that only a small quantity of DNA contained in the solution 64 is carried forward to the following stage.

In the amplification stage 18, the solution 64 is mixed with a master solution 66. Approximately 20 μl of master solution 66 is used together with the 1-5 μl of solution 64. The master solution 66 comprises a buffer, an enzyme—Taq DNA polymerase, two oligonucleotide primers, deoxynucleoside triphosphate (dNTPs) and a cofactor, MgCl₂. The primers determine which region of the DNA material is to be amplified.

In the amplification stage 18, the solution, being a combination of the solutions 64 and 66, is heated firstly to a temperature in a range of about 94-96° C., preferably 94° C., for 30 seconds to denature the target DNA. The temperature is lowered to a temperature in a range of about 50-65° C., preferably about 55° C., for a further 30 seconds to permit the primers to anneal to their complementary sequences. Finally, the temperature is raised to a temperature of about 72° C. for a further 30 seconds to allow the Taq DNA polymerase to attach at each primed site and to form a new DNA strand. The cycling through the various temperatures is repeated approximately 30 times so that the DNA material is multiplied more than a billion times.

The amplified solution 68 is fed from the amplification stage 18 to the purification stage 22. Once again, approximately 1-5 μl of solution 68 is fed through the purification stage 22. The purification stage 22 comprises a gel filtration device 70. The filtration device 70 is in the form of a tube 72 containing a quantity of gel filtration medium 74. A valve 76 controls the passage of the solution 68 through the tube 72. A waste valve 78 is provided through which waste material can be discharged to a container 80 to remove the dNTPs, primers and reaction products other than the material of interest.

In the purification stage, the gel filtration medium 74 allows the larger fragments of DNA through before allowing any smaller fragments, dNTPs and primers through.

A suitable gel filtration medium is a resin composed of macroscopic beads synthetically derived from the polysaccharide, dextran, such as that sold under the trade name, Sephadex G50/G25 (Sephadex is a registered trade mark of Amersham Biosciences AB, Uppsala, Sweden).

The larger fragments of DNA are collected at the downstream end of the tube 72 for sequencing in the sequencing stage 20.

In the sequencing stage 20, the DNA in the solution 82 is sequenced into many pieces of differing lengths using restriction enzymes. Each piece is used as a template to generate a set of DNA fragments where any one DNA fragment differs in length from any other DNA fragment by a single nucleotide base.

The nucleotide base at the end of each of the DNA fragments is tagged with one of four dideoxynucleoside bases (ddATP, ddTTP, ddCTP, ddGTP). Since each of the four nucleoside bases contains a different dye, when excited with a laser,.the bases emit light at different wavelengths. For this purpose, the system 10 has a supply 84 of a solution containing dyes which is fed into the thermal cycler to effect sequencing. A suitable sequencing solution that can be used is Big-Dye (Big-Dye is a trade mark of Applied Biosystems, USA). The sequencing solution is mixed in a quantity of about 20 μl with the solution 82 to dye the nucleotide bases at the ends of the DNA fragments.

To randomly terminate the nucleotide bases and fluorescently label the ends of the DNA fragments, the solution in the thermal cycler 12 is cycled through a temperature of approximately 96° C. for about 30 seconds followed by a temperature of approximately 50° C. for about 15 seconds followed by a temperature of approximately 60° C. for about 4 minutes. This cycle is repeated approximately 25 times.

The solution 86 with the fluorescently labelled DNA fragments is fed from the thermal cycler 12 into a separation stage of an analysis stage 88 of the system 10. The separation stage makes use of electrophoresis equipment, more particularly, capillary electrophoresis equipment 90. The equipment 90 includes a capillary 92, containing polyacrylamide or agarose gel, having an upstream end in a sample vial 94 into which the fluorescently labelled DNA fragments are fed from the sequencing stage 20. The DNA fragments are fed through the capillary 92 into an output vial 96. As the solution 86 moves through the capillary 92, the solution 86 is subjected to a high voltage field provided by a high voltage power supply 98. The power supply 98 provides a voltage in the region of 5-30 kV. Because the DNA fragments are of different lengths, they take different amounts of time to migrate from one end of the capillary 92 to the other end.

The analysis stage 88 of the system 10 includes a detecting stage, or detector, 110 for detecting and reading the nucleotide bases of the DNA fragments. The detector 110 comprises an excitation source in the form of a laser 100 to excite the fluorescently labelled ends of the DNA fragments. Thus, the DNA fragments passing through the capillary 92 are subjected to laser light from the laser 100. The detector 110 further includes a reader in the form of a CCD camera 102, and/or a spectrograph or one or more photomultiplier tubes (PMTs) for reading the wavelength of the fluorescing material. An output 104 from the camera 102 is fed to the computer 14 where an electropherogram, 106 is displayed on a screen 108 of the computer 14 representative of the DNA sequence of the sample 60. Software of the computer converts the collected data into sequence information using a base-calling algorithm to produce the electropherogram. The electropherogram is a plot of sequence data.

It will be appreciated that the electropherogram 106 is generated by reading off the light from a fmal nucleotide base at the end of each DNA fragment. Since each base is tagged with a different colour, it is possible to detect the order of the nucleotide bases in the DNA fragment sequence.

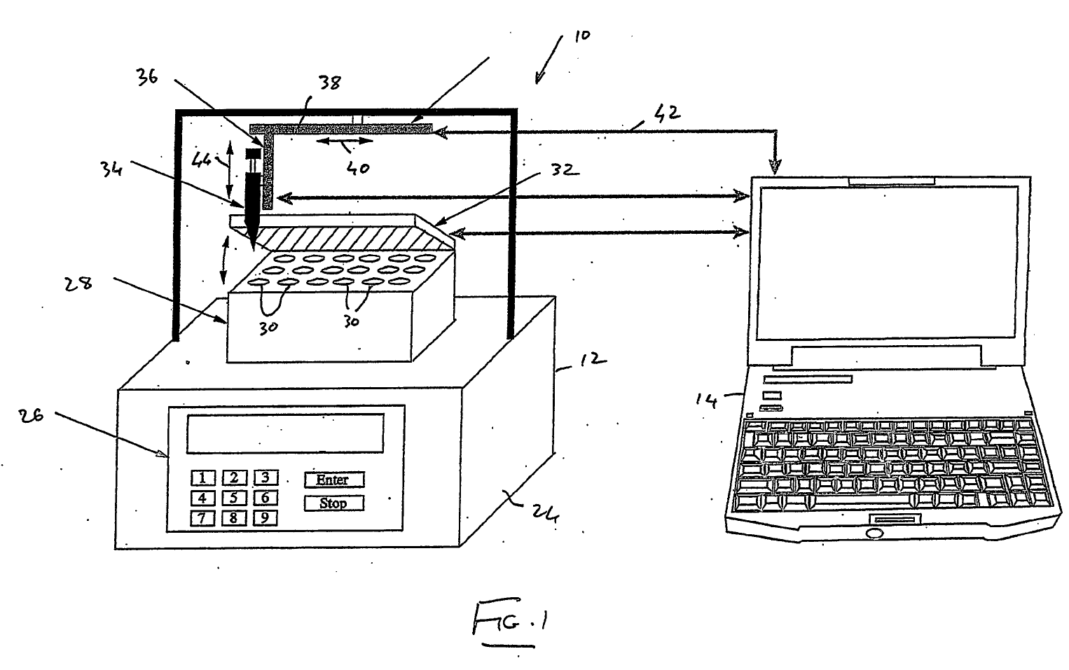

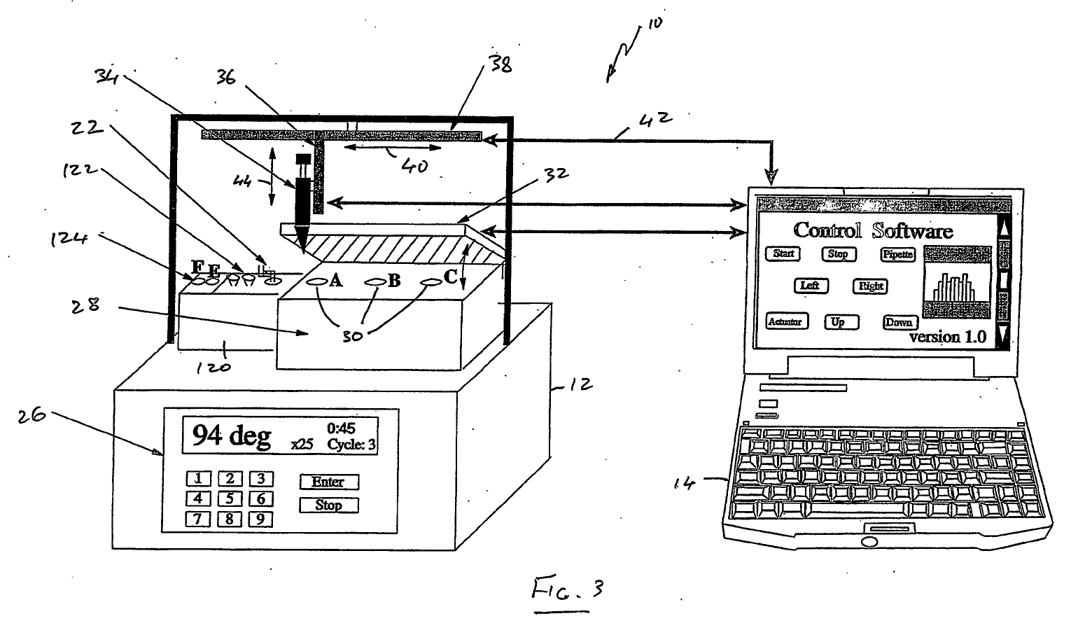

Referring to FIG. 3 of the drawings, a modified DNA analysis system is illustrated. With reference to FIGS. 1 and 2 of the drawings, like reference numerals refer to like parts, unless otherwise specified.

In this embodiment of the invention, a holder 120 is arranged alongside the heat block 28. The holder 120 holds a set of replaceable plastics tips 122 for the pipette 34.

It is to be noted that the holder 120 is positioned alongside the heat block 28 to be within the range of movement of the pipette 34 horizontally in the direction of the arrows 40 and vertically in the direction of the arrows 44.

The holder 120 further defines a plurality of reservoirs 124. The solutions for use in the amplification stage and in the sequencing stage, i.e. the PCR solution and the Big-Dye solution, respectively, are contained in the reservoirs 124. These reservoirs 124 are also within the range of movement of the pipette 34. Therefore, solutions from the reservoirs 124 can be added to the wells 30 containing the sample 60.

In this embodiment, pre-prepared solutions 66 and 84 are deposited in the reservoirs 124. The samples 60 along with the thermo-stable proteinase and buffer are added to the well 30A. The heat lid 32 is closed and the thermal cycler 12 carries out the pre-programmed temperature profile to effect extraction. This procedure takes approximately 45 minutes and, once it has been completed, the lid 32 is automatically opened under the control of the computer 14. Between 1 and 5 μl of the solutions 64 is transferred to the well 30B.

The pipette 34, after having had its tip 122 replaced if necessary, collects solution 66 from one of the reservoirs 124 and deposits it in the well 30B of the heat block 28. The lid 32 of the thermal cycler 12 is again closed and the cycling protocol for the amplification reaction is carried out in a time period of about 40 minutes.

Upon completion of amplification, the lid 32 is opened, the solution is extracted from the well 30B by the pipette 34 and is deposited in the purification stage 22 which, as shown, is also mounted on the holder 120. After purification, the solution is removed from the purification stage 22 by the pipette 34 and is deposited in well 30C together with Big Dye solution collected by the pipette 34 from the appropriate reservoir 124 and which is also deposited in the well 30C.

The lid 32 is again closed and the sequencing reaction is performed by cycling through the relevant temperature profile. The solution is then available for analysis in a sequencer.

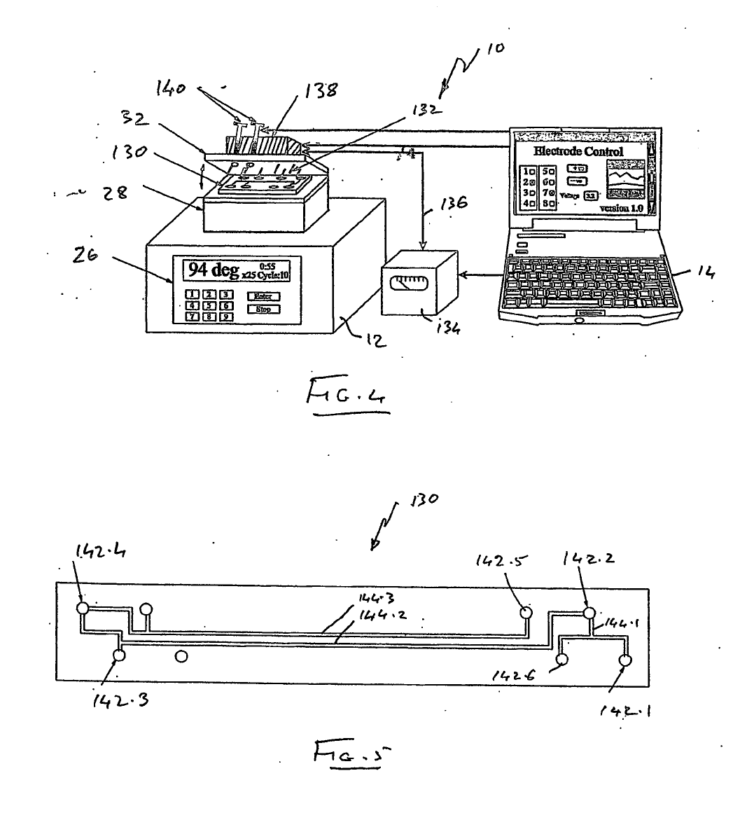

Referring to FIGS. 4 and 5 of the drawings, a further embodiment of the invention is illustrated. Once again, with reference to the previous drawings, like reference numerals refer to.like parts, unless otherwise specified.

In this embodiment of the invention, instead of the heat block 28 containing the wells 30, a microfluidic device in the form of a microfluidic chip 130 is mounted on the heat block 28 of the thermal cycler 12. The extraction, amplification, purification and sequencing stages of the DNA analysis system 10 are carried out in the microfluidic chip 130.

The system 10 includes an electric field generating means in the form of a plurality of electrodes 132 connected to a power supply 134 via a line 136 and an electrode control unit 138 mounted on the lid 32.

Also, to dispense liquid or solution into the wells of the microfluidic chip 130, as will be described in greater detail below, a plurality of external pipettes 140 are arranged on the lid 32.

A plan view of the microfluidic chip 130 is shown in greater detail in FIG. 5 of the drawings. The microfluidic chip 130 used by the Applicant is a Protolyne™ semi-custom microfluidic chip Protolyne is a Trade Mark of Micralyne Inc., Alberta, Canada). The chip 130 is fabricated using MEMS technology and consists of two glass plates in which wells 142 are etched. The chip 130 is pre-fabricated with the wells 142 in position but channels 144 can be etched as required.

Hence, as shown, the chip 130 comprises eight wells 142 and was etched with the pattern of channels 144 as shown in FIG. 5 of the drawings.

A first well 142.1 of the chip 130 is used as an extraction well, a second well 142.2 is used as an amplification well, a third well 142.3 is used as a waste well and a fourth well 142.4 is used as a sequencing well. A fifth well 142.5 is available for the capillary electrophoresis stage.

As an initial step, sieving material was deposited in the channel 144.1 interconnecting the wells 142.1 and 142.2 as well as in the channel 144.2 interconnecting the wells 142.2 and 142.4. In this regard, it is to be noted that a channel 144.3 interconnects the wells 142.4 and 142.5 to enable the final step of capillary electrophoresis to be effected.

The sample 60 to be analysed is deposited into the well 142.1 together with the extraction reagents, as described above. Once extraction has been completed, the next step is to effect amplification by PCR Accordingly, at the end the extraction procedure, and due to the fact that a DNA sample is negatively charged, a negative voltage is applied by the relevant electrode 132 to the extraction well 142.1. The amplification well 142.2 is kept at ground voltage. The application of the negative voltage to the well 142.1 expels the solution from the well 142.1 into the channel 144.1. The sample 60, in solution, moves towards the amplification well 142.2 but, due to capillary action, does not enter the well 142.2.

Once the solution is in the channel 144.1, a positive voltage is applied to the amplification well 142.2 using the relevant electrode 132. The extraction well 142.1 is maintained at zero voltage. This creates a positive voltage gradient resulting in the solution being deposited in the amplification well 142.2. Once the required quantity of solution has been deposited into the well 142.2, control of the voltages can be discontinued. Any superfluous solution can be deposited in a well 142.6.

Prior to sequencing the solution in the well 142.4 it needs to be purified to remove contaminants, as described above. This purification is done by applying a positive voltage to the waste well 142.3 while keeping the amplification well 142.2 grounded. Since the channel 144.2 contains a sieving matrix and because the amplified DNA molecules are of a different size and have different electrophoretic mobilities in comparison with the contaminants, they will migrate across the channel 144.2 at different rates. Because the DNA molecules are larger in size and take longer to move through the channel 144.2, the contaminants will move through the channel 144.2 ahead of the DNA molecules.

Accordingly, after applying the positive voltage to the waste well 142.3 for a short period of time, the contaminants migrate and are defused into a buffer present in the waste well 142.3 while the DNA molecules are contained in the channel 144.2.

The positive voltage applied to the waste well 142.3 is discontinued and, instead, a positive voltage is applied to the sequencing well 142.4 to attract the DNA molecules in the channel 144.2 into the sequencing well 142.4 for sequencing purposes. The required sequencing reagents are added to the well 142.4 using one of the pipettes 140.

An advantage of using the microfluidic chip 130 is a further reduction in size of the system 10 to effect extraction, amplification, purification and sequencing of the sample.

Typically, to effect movement of the fluid between the wells, a voltage of−2kV or +2kV, as the case may be, is applied for predetermined periods of time. For example, to effect movement of the solution from the extraction well 142.1 into the channel 144.1 involves applying a voltage of −2kV for approximately 20 seconds. To effect movement of the solution from the channel 144.1 into the amplification well 142.2 involves the application of a voltage of +2kV to the amplification well 142.2 for a period of about 2.5. minutes to 3 minutes.

Because of the use of the proteinase, as defined above, a system 10 is provided which makes use of the thermal cycler 12 for effecting extraction, amplification, and sequencing using a single device. Hence, a portable, field-useable, system 10 is provided which requires minimum human intervention. More particularly, the need to open the test tubes or wells 30 containing the sample material 60 regularly is obviated thereby reducing the risk of contaminating the sample material 60 to be analysed.

Still further, because the proteinase is denatured in the extraction phase, it is not necessary to make use of separating equipment such as centrifuges. This further reduces the size and weight of the system 10 rendering it portable.

Hence, it is a particular advantage of the invention that a portable DNA analysis system is provided. The system is integrated and requires very little human intervention or expertise to operate. The benefit of an integrated system is a reduction in the number of components and also the costs of conducting the analysis by reducing the labour costs and sample reagent consumption.

Such a system is particularly useful in fields such as health care, agriculture, forensic medicine, military applications, environmental monitoring, animal husbandry, or the like. The use of a portable system provides the ability for analysis to be done in situ with the resultant, self-evident advantages.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A DNA analysis system which includes a unit that effects both extraction of DNA and amplification by identical replication of a region of interest of extracted DNA strands, with a proteinase, as defined, being used in the unit at least to effect extraction of DNA.

2. The system of claim 1 in which the amplification includes nucleotide sequence detection for the purpose of looking for specific sequences of DNA.

3. The system of claim 2 in which the unit includes an attached fluorimeter and light source.

4. A DNA analysis system which includes:
a thermal cycler operable as an extraction stage for extracting DNA from a sample to be tested and as an amplification stage for replicating identically a region of interest in DNA strands extracted from the sample, a proteinase, as defined, being used in the thermal cycler at least in the extraction stage;
a purification stage for purifying the amplified material from the thermal cycler; and
an analysis stage for analysing the purified sample to obtain genetic information relating to the sample.

5. The system of claim 4 in which the analysis stage comprises a separation stage and a detection stage.

6. The system of claim 4 which includes a sequencing stage preceding the analysis stage.

7. The system of claim 6 in which the thermal cycler is used for the sequencing stage.

8. The system of claim 6 in which the purification stage incorporates a size filtration matrix comprising a gel filtration media incorporating a filtering resin, the matrix allowing larger fragments of DNA through from the amplification stage before any smaller fragments and other unwanted substances.

9. The system of claim 8 in which the larger fragments are collected for use in the sequencing stage.

10. The system of claim 9 in which the sequencing stage tags ends of the fragments with dideoxynucleoside triphosphates (ddNTP's) labelled with different fluorochromes before grading.

11. The system of claim 10 in which the grading forms the first step of the separation stage and incorporates separating the fragments into fragments of differing lengths by a separation device.

12. The system of claim 11 in which the separation device is an electrophoresis device.

13. The system of claim 12 in which the electrophoresis device is a capillary electrophoresis device and includes a detector for detecting information relating to tagged fluorescent nucleotides at the end of each of the DNA fragments.

14. The system of claim 13 in which the detector includes a laser device that irradiates the ends of the DNA fragments to cause the fluorescent ends to fluoresce.

15. The system of claim 14 which includes a reader for reading the fluorescent ends of the fragments.

16. The system of claim 4 in which the thermal cycler includes a controller which controls the various stages of preparation of the sample.

17. The system of claim 16 in which the thermal cycler includes a heating mechanism for heating the sample, contained in one or more vials or test tubes, received in the thermal cycler.

18. The system of claim 17 in which the heating mechanism is controlled by the microcontroller to maintain the sample at the required temperatures at the various stages of extraction, amplification and sequencing.

19. The system of claim 17 which includes a dispensing device for depositing the material to be analysed in the thermal cycler.

20. The system of claim 19 in which the thermal cycler includes a holder for holding replacement tips for the dispensing device.

21. The system of claim 20 in which the holder is arranged on the thermal cycler adjacent the heating mechanism within reach of the range of movements of the dispensing device.

22. The system of claim 21 in which the holder includes reservoirs for various solutions adjacent the replacement tips.

23. The system of claim 20 in which the purification stage is mounted on the holder adjacent the heating mechanism of the thermal cycler.

24. The system of claim 4 which includes a monitoring means for monitoring the analysis stage.

25. The system of claim 24 in which the monitoring means is in the form of a computer having a display on which data relating to the analysed sample are displayed.

26. A method of preparing a sample for DNA analysis, the method including the step of using a single unit to effect both extraction of DNA and amplification by identical replication of a region of interest of extracted DNA strands, with a proteinase, as defined, being used in the unit at least to effect extraction of DNA.

27. The method of claim 26 which includes the step of looking for specific sequences during amplification by including nucleotide sequence detection in the amplification stage.

28. The method of claim 27 which includes performing nucleotide sequence detection during amplification by adding fluorescently labelled oligonucleotides that can target a specific sequence of DNA.

29. The method of claim 28 which includes using a thermal cycler that has an attached fluorimeter and light source.

30. A method of preparing a sample for DNA analysis, the method including the steps of:
placing a sample of material to be analysed in a thermal cycler and adding a predetermined quantity of proteinase to the thermal cycler;
cycling the mixture through a predetermined temperature profile to effect extraction of DNA material from the sample;
in the thermal cycler, subjecting the extracted DNA material to an amplification stage replicating identically a region of interest in the extracted DNA material; and
sequencing the amplified material.

31. The method of claim 30 which includes sequencing the material by a dideoxy method of sequencing which includes the steps of sequencing, separation and detection.

32. The method of claim 30 which includes, as part of separating the DNA material, purifying the material and sequencing the purified DNA material.

33. The method of claim 32 which includes effecting the sequencing of the purified DNA material for separation and detection using the thermal cycler.

34. The method of claim 32 which includes purifying the material by passing the material through a size filtration matrix comprising a gel filtration media incorporating a filtering resin, the matrix allowing larger fragments of DNA through from the amplification stage before any smaller fragments and other unwanted substances.

35. The method of claim 34 which includes collecting the larger fragments for use in the sequencing of the material.

36. The method of claim 35 which includes tagging ends of the fragments with dideoxynucleoside triphosphates (ddNTP's) labelled with different fluorochromes before grading.

37. The method of claim 36 in which the grading forms the first step of the separation stage and the method incorporates separating the fragments into fragments of differing lengths.

38. The method of claim 36 which includes detecting information relating to tagged fluorescent nucleotides at the end of each of the DNA fragments.

39. The method of claim 38 which includes irradiating the ends of the DNA fragments to cause the fluorescent ends to fluoresce and reading the fluorescent ends of the fragments.

40. A purification stage for a DNA analysis system, the purification stage including
a conduit; and
a gel filtration medium contained in the conduit, the gel filtration medium being a resin of microscopic, synthetic beads.

41. The purification stage of claim 40 in which the gel filtration medium is of microscopic beads synthetically derived from a polysaccharide.

42. The purification stage of claim 41 which includes a control device for controlling the passage of the sample through the conduit.

43. A method of purifying a DNA sample, the method including the step of passing the sample through a conduit containing a gel filtration medium in the form of a resin of microscopic, synthetic beads to effect purification of the sample.

44. The method of claim 43 which includes forming the beads from a polysaccharide.

45. The method of claim 43 which includes controlling the passage of the sample through the conduit.

46. A DNA analysis system which includes:
a unit operable at least as an extraction stage for extracting DNA from a sample to be tested and as an amplification stage for replicating identically a region of interest in DNA strands extracted from the sample;
a microfluidic device mounted on the unit and defining a plurality of wells interconnected by a channel, a sample undergoing various stages of preparation being moved sequentially from one well to another via the relevant interconnecting channel; and
a control arrangement for controlling movement of the sample between said wells.

47. The system of claim 46 in which the unit also operates as a sequencing stage.

48. The system of claim 46 in which the control arrangement includes an electric field generating means that moves a charged solution between the wells through the channels.

49. The system of claim 48 in which the electric field generating means comprises a plurality of electrodes, each of said predetermined wells having an electrode associated with it.

50. The system of claim 46 in which at least certain of the wells operate as waste wells in which waste material, separated out from the sample, is deposited for disposal.

51. The system of claim 46 which includes a dispensing arrangement for depositing reagents in the wells.

52. The system of claim 51 in which the dispensing arrangement comprises at least one pipette for dispensing the reagents.

53. A method of preparing a sample for DNA analysis, the method including the steps of:
placing a sample of material to be analysed in a first well of a microfluidic device having a plurality of wells interconnected by channels;
effecting a first preparatory stage in the first well of the device;
controlling movement of the sample from one well, sequentially, to further wells in the microfluidic device and carrying out further preparatory stages at each of predetermined wells in the device.

54. The method of claim 53 which includes modifying an existing thermal cycler by mounting the microfluidic device on the thermal cycler.

55. The method of claim 53 which includes controlling the movement of the sample from well to well by means of an electric field generating means that moves a charged solution between the wells through the channels.

56. The method of claim 55 which includes associating an electrode with each well and controlling the movement of the sample between wells by changing the potential of the wells relative to one another.

57. The method of claim 53 which includes designating one of the wells as a waste well and depositing waste material, separated out from the sample, in the waste well.
